(19) United States
(12) Patent Application Publication
(10) Pub. No.: US 2023/0241177 A1
(43) Pub. Date: Aug. 3, 2023

(54) USE OF INHALED INTERFERON-BETA TO TREAT VIRUS-INDUCED EXACERBATIONS IN COPD PATIENTS UNDERGOING TREATMENT WITH A SYSTEMIC CORTICOSTEROID

(71) Applicant: SYNAIRGEN RESEARCH LIMITED, Southampton Hampshire (GB)

(72) Inventors: Richard MARSDEN, Southamptom Hampshire (GB); Phillip MONK, Southamptom Hampshire (GB); Victoria TEAR, Southamptom Hampshire (GB); Tom WILKINSON, Southamptom Hampshire (GB)

(21) Appl. No.: 18/024,648

(22) PCT Filed: Sep. 7, 2021
(86) PCT No.: PCT/GB2021/052302

§ 371 Date: Mar. 3, 2023

(30) Foreign Application Priority Data
Sep. 8, 2020 (GB) 2014114.9

(51) Int. Cl. A61K 38/21; A61K 9/00; A61P 11/00

(52) U.S. Cl. CPC A61K 38/215; A61K 9/0073; A61P 11/00

(57) ABSTRACT

The present invention provides interferon-beta (IFN-β) for use in the treatment of virus-induced COPD exacerbations in patients treated with a systemic corticosteroid, wherein the IFN-β is administered by inhalation, e.g. by use of a nebuliser.

### FIELD OF THE INVENTION

The present invention relates to the use of inhaled interferon-beta (IFN-β), e.g. formulated for nebuliser administration via the airways, to treat COPD patients whose condition is exacerbated by viral infection and who are also undergoing treatment with a systemic corticosteroid.

### BACKGROUND TO THE INVENTION

Chronic Obstructive Pulmonary Disease (COPD) is a lung condition characterised by airflow limitation that is not fully reversible. This airflow limitation is normally progressive and is associated with an abnormal inflammatory response of the lung to pathogenic stimulus. The majority of COPD is associated with long-term cigarette smoking. Symptoms of COPD include cough, excessive sputum production and shortness of breath.

Exacerbations of COPD are defined as the worsening of COPD symptoms beyond normal day-to-day variations and are associated with irreversible loss of lung function and therefore accelerated disease progression. Exacerbations severely impact on the patient's quality of life (patients typically take a number of weeks to recover) and are a major healthcare burden. Exacerbations are currently treated with systemic corticosteroids and/or antibiotics. Systemic treatments include oral medicines (given by mouth) or medicine that is delivered directly into a vein (intravenously or IV) or muscle (intramuscularly). Systemic corticosteroids circulate through the bloodstream to various body sites.

Respiratory viral infections, such as the common cold and flu, are a major driver of exacerbations in patients with lung disease when infections spread from the upper respiratory tract to the lungs to worsen pre-existing lung inflammation. Furthermore, particularly in COPD, there is growing evidence that virus infections increase susceptibility to follow-on bacterial infections. Therefore, there is strong rationale to develop anti-viral treatments to prevent or treat exacerbations of COPD.

In pre-clinical studies, researchers have found that lung cells from COPD patients and/or long term smokers are more susceptible to infection with respiratory viruses, explaining why infections may be more likely to spread to the lungs (Schneider et al. (2010) Am. J. Respir. Crit. Care Med. 182(3), 332-340). Interferon-beta (IFN-β) is produced by cells, of particular relevance, lung epithelial cells in response to viral infection and orchestrates the body's anti-viral responses. In further experiments, IFN-β pre-treatment has been found to protect lung cells from COPD patients against infection with a range of respiratory viruses that are associated with exacerbations of COPD.

Deficiencies in IFN-β-mediated anti-viral responses have also been associated with a worse outcome in virus challenge studies conducted in COPD and COPD patients who have more frequent exacerbations (Hilzendeger et al. (2016) Int. J. Chron. Obstruct. Pulmon. Dis. 11, 1485-1494; Mallia et al. (2011) Am. J. Respir. Crit. Care Med. 183(6), 734-742).

IFN-β driven anti-viral responses have been shown to be compromised/deficient in older people and those with chronic airways diseases, more particularly asthma and COPD (Agrawal et al. (2013) Gerontology 59, 421-426; Wark et al. (2005) J. Exp. Med. 201(6): 937-47; Singanavagam et al. (2019) Am. J. Physiol. Lung Cell Mol. Physiol. 317(6): L893-L903). This is in keeping with previous proposed use of inhaled IFN-β to treat virus-induced exacerbations of asthma and chronic obstructive pulmonary disease (COPD) caused by common cold-causing viruses, such as rhinoviruses (see EP1734987B in the name of University of Southampton and exclusively licensed to Synairgen plc) and the previous proposed use of inhaled IFN-β to reduce severity of LRT illness in the elderly arising from common cold-causing viruses, such as rhinovirus infection (See U.S. Pat. No. 7,871,603B in the name of Synairgen Research Limited). Additionally, EP2544705B, also in the name of Synairgen Research Limited, proposes use of inhaled IFN-β for treatment of LRT illness associated with influenza infection.

Clinical trials using an inhaled IFN-β formulation for nebulisation delivered via a breath-actuated nebuliser have been conducted to further such administration especially in asthmatics or COPD patients suffering LRT illness through a cold or influenza with encouraging results. In all such clinical trials (3 in asthma and one in COPD patients) conducted to date, inhaled IFN-β has upregulated lung antiviral biomarkers in sputum for 24 hours after dosing (Djukanović et al (2014) Am. J. Respir. Crit. Care Med. 190(2):145-54) confirming successful delivery of biologically active drug to the lungs, demonstrating proof-of-mechanism, and supporting dose selection.

Inhaled corticosteroids help reduce inflammation in the airways and are widely used as part of a combination therapy for COPD patients, especially those with a history of exacerbations. Indeed, Singanayagam et al. (Nature Communication (2018) 9:2229) reported that inhaled corticosteroids suppressed inflammation and immune responses in mice infected with rhinovirus. However, this was also associated with impaired lung virus control, increased mucus production and deficient antimicrobial peptide responses. It was also shown that inhaled corticosteroid suppressed induction of IFN-β and that the reduction in immune response could be ameliorated by recombinant IFN-β administration. The same study also reported that, at exacerbation, IFN-β mRNA levels were reduced in sputum from COPD patients taking inhaled corticosteroids, yet paradoxically at the same time, expression of several antiviral genes was elevated and there was no significant difference in levels of these biomarkers between patients who were treated without or with inhaled corticosteroids. However, two weeks after the onset of the exacerbation, IFN-β levels had returned to baseline, but antiviral gene expression remained elevated only in the group that were not treated with inhaled corticosteroids. It was hypothesised by Singanayagam et al. that inhaled IFN-β may have a protective effect by replacing the endogenous IFN-β whose production had been suppressed by the action of inhaled corticosteroid therapy and that this effect should be studied further, although the actual effect of recombinant IFN-β on virally-infected COPD patients was not investigated. While the study by Singanayagam et al. is consistent with other studies that demonstrate the ability of corticosteroids to suppress IFN-β production (McCoy C. E. et al, J. Biol. Chem. 2008; 283(21); 14277-14285), it is also known that corticosteroids can affect downstream signalling from the Type I interferon receptor (Diez D. et al BMC Med Genomics 2012; 5; 27 and Flammer J. R et al Mol Cell Biol 2010; 30(19): 4564-4572). It is not known whether these two effects are equally sensitive to inhibition by corticosteroids and importantly, it is not known how much IFN-β might be required to overcome the inhibitory effect of corticosteroids on IFN-signalling pathways.

While inhaled corticosteroids are used as a controller medication for some COPD patients, when COPD patients suffer an acute exacerbation, current guidelines recommend that, in the absence of significant contraindications, the dose of corticosteroid should be increased, usually by giving oral corticosteroids whether these patients are admitted to hospital or are in the community (https://www.nice.org.uk/guidance/ng115/chapter/recommendations#systemic-corticosteroids). As the impact of systemic corticosteroid medication was not investigated in the study by Singanayagam et al., it has remained unknown whether inhaled IFN-β can have any benefit in preventing or treating COPD in patients whose disease is acutely exacerbated by viral infection and who are also being treated with a systemic corticosteroid. While the preclinical studies of Singanayagam et al. might suggest that such an approach is reasonable, Ranieri et al. (JAMA, 2020:323(8):725-733 published 17 Feb. 2020) reported the results of the INTEREST clinical trial which investigated the effect of intravenous IFN-β on mortality and days free from mechanical ventilation in patients with moderate to severe acute respiratory distress syndrome (ARDS). The results showed that in adults with moderate to severe ARDS intravenous administration of IFN-β had no significant benefit compared to placebo on number of deaths and number of ventilator-free days over 28 days. While the results did not support the use of IFN-β in the management of ARDS, post-hoc analysis of the study identified a treatment benefit in patients that received IFN-β but were not treated with systemic corticosteroids, when compared to those treated with both IFN-β and systemic corticosteroids (Jalkanen J et al. Intensive Care Med. 19: 1-4, 2020; published May 2020). In the same paper, this difference was explained by ex vivo studies with human lung tissue or human primary lung endothelial cells where corticosteroid treatment prevented IFN-signalling and expression of STAT1, IRF9 and cluster of differentiation 73 (CD73) by IFN-β; the latter biomarker is a key molecule preventing vascular leakage and harmful leukocyte infiltration into the lungs in ARDS patients. Both studies cautioned that the use of IFN-β in patients on systemic corticosteroid should be considered carefully due to the ability of corticosteroid to inhibit IFN-β signalling. While Ranieri et al. noted that this caution is not only in relation to patients suffering with ARDS but all studies in which IFN-β is administered in conjunction with systemic corticosteroids, Jalkanen et al highly recommended not to use systemic glucocorticoids together with type I interferons because of the harmful effects of this combination.

Thus, there are conflicting views in the prior art as to whether IFN-β therapy is a viable treatment option for COPD patients undergoing systemic corticosteroid therapy. Indeed, the state of the art strongly cautions against IFN-β therapy in this context.

Now presented for the first time are data supporting the use of inhaled IFN-β to treat COPD patients whose condition is exacerbated by viral infection and who are also undergoing treatment with a systemic corticosteroid.

### SUMMARY OF THE INVENTION

Thus, the present invention provides interferon-beta (IFN-β) for use in the treatment of virus-induced COPD exacerbations in patients treated with a systemic corticosteroid, wherein the interferon-beta is administered by inhalation.

“Treating” or “treatment” in the context of the present invention is understood to relate to an improvement in lung function or symptoms, or prevention of secondary bacterial infections in COPD patients who are being treated with a systemic corticosteroid for a virus-induced exacerbation of their disease. This may be assessed by improvements in lung function parameters, such as Peak Expiratory Flow Rate (PEFR), breathlessness, cough, sputum production or purulence (associated with bacterial infections), or the prevention of worsening symptoms driven by secondary bacterial infections.

The invention will be hereinafter principally described with reference to common respiratory viruses in humans, and their exacerbation of COPD-related symptoms. The invention is seen by reasonable extrapolation as having application to any known virus that infects the airways of the respiratory tract to cause symptoms of the common cold. Therefore, in some embodiments the virus that causes exacerbation of COPD symptoms may be rhinovirus, influenza, RSV, adenovirus, parainfluenza, human metapneumovirus or coronavirus. Coronavirus in the context of the present disclosure is understood to mean the types of coronavirus that typically cause common cold symptoms but not to highly pathogenic coronaviruses such as SARS-CoV, MERS-CoV, or SARS-CoV-2, the virus that causes COVID-19.

Thus in its broadest aspect, the present invention may be seen as providing IFN-β for use in reducing the severity of virus-induced exacerbations in COPD patients treated with systemic corticosteroids, wherein the IFN-β is administered by inhalation.

Systemic corticosteroids, typically delivered via the oral or injected route of administration, are widely used in the treatment of acute exacerbations of COPD in order to reduce symptoms associated with inflammation of lung tissue. The corticosteroid may be selected from prednisolone, hydrocortisone, dexamethasone, methylprednisolone or prednisone or combinations thereof.

Systemic corticosteroids in the context of the present invention are understood to relate to corticosteroids that are administered to the patient such that they act throughout the patient's body and not locally at a specific point or area of the body.

The aim of treatment with inhalable IFN-β is to reduce the symptoms of a virus-induced exacerbation in COPD patients treated systemically with corticosteroids. The mechanism of action may be because the recombinant IFN-β delivered to the patient overcomes an inhibitory action of corticosteroid on induction of IFN-β gene expression and/or suppression of IFN-β driven antiviral responses or by the delivered IFN-β overcoming a lack of naturally expressed IFN-β in the patient, for example due to the patient's age or inability to produce IFN-β.

Effectiveness of the inhaled IFN-β may be monitored in terms of improvement of lung function or symptoms, for example, by assessing Peak Expiratory Flow Rate (PEFR), a measure of lung function.

PEFR is a person's maximum speed of expiration, as measured with a peak flow meter. This is a hand-held device used to monitor a person's ability to breathe out air. It measures the airflow through the bronchi and thus the degree of obstruction in the airways. Patients are able to measure their own PEFR before and after taking any COPD medication and/or before or after taking inhalable IFN-β as used in the present invention. An example of a suitable PEFR monitor is the eMini-Wright, Digital Peak Flow Meter (model. 3210001) made by Clement Clarke International.

COPD patients with a virus-induced exacerbation treated with systemic corticosteroid and/or antibiotics and treated with inhalable IFN-β according to the present invention are typically 50 years of age or older and/or have improved breathlessness following treatment as determined by the BCSS test (Breathless, Cough and Sputum Scale). Typically, the patients are in the age range of from 40 to 90 years. Preferably the age range may be from 60 to 85 years. The lower end of the age range may be 40, 50, 60, 70 or 80 years. The upper end of the age range may be 90, 80, 70, 60 or 50 years. Any combination of these lower and upper end ranges is contemplated by the present invention. Patients having received treatment according to the present invention typically have a Forced Expiratory Volume (FEV) in one second of 55 to 65%.

The BCSS test is a patient-reported outcome measure in which patients are asked to record the severity of three symptoms: breathlessness, cough and sputum.

Each symptom is represented by a single item which is evaluated on a 5-point scale ranging from 0-4, with higher scores indicating more severe symptoms. Total score is expressed as the sum of the three-item score, with a range of 0-12. A mean decline of 1 point on the BOSS total scale signifies a substantial reduction in symptom severity.

This assessment is typically carried out once a day at the same time each day (+1-3 hours).

The BOSS questions and possible responses are as follows:
1. How much difficulty did you have breathing today?
0=None—unaware of any difficulty
1=Mild—noticeable when performing strenuous activity (e.g. running)
2=Moderate—noticeable even when performing light activity (e.g. bedmaking or carrying
groceries)
3=Marked—noticeable when washing or dressing
4=Severe—almost constant, present even when resting
2. How was your cough today?
0=No cough—unaware of coughing
1=Rare—cough now and then
2=Occasional—less than hourly
3=Frequent—one or more times an hour
4=Almost constant—never free of cough or need to cough
3. How much trouble did you have due to sputum today?
0=None—unaware of any trouble
1=Mild—rarely caused trouble
2=Moderate—noticeable trouble
3=Marked—caused a great deal of trouble
4=Severe—almost constant trouble

The invention is described further below by reference to the clinical trial data provided in the exemplification and illustrated by the figures as described below.

### DESCRIPTION OF THE FIGURES

FIG. 1: Upregulation of gene expression of the IFN-β dependent antiviral biomarker MX1 in lung (sputum) cells, 24 hours after administration of inhalable IFN-β, demonstrates that inhalable IFN-β can switch on antiviral defences in the lungs of COPD patients, who were not receiving systemic corticosteroids.

FIG. 2: Gene expression of the IFN-β dependent antiviral biomarker MX1 was upregulated in lung (sputum) cells to a similar extent in response to inhalable IFN-β in COPD patients whether they were treated with systemic corticosteroids and/or antibiotics for an exacerbation (Group B) or not (Group A). This suggests that inhalable IFN-β can boost IFN signalling and antiviral responses even in the presence of systemic corticosteroids, and thus has potential to treat COPD patients who are receiving a systemic corticosteroid for a virus-induced exacerbation of their disease.

FIG. 3: Inhalable IFN-β significantly improved lung function (PEFR) in COPD patients treated with systemic corticosteroids for a virus-induced exacerbation. The difference in change from baseline PEFR over the treatment period (days 2 to 15) between patients receiving inhalable IFN-β and placebo was 25.5 L/min (95% Cl 1.1, 49.9; p=0.04). These data demonstrate that IFN-β therapy is a viable treatment option for COPD patients undergoing systemic corticosteroid therapy.

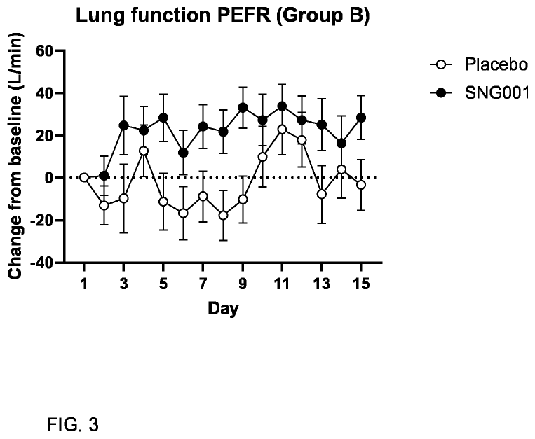

### DETAILED DESCRIPTION

Nature of Interferon Beta for Administration

The term IFN-beta or IFN-β as used herein will be understood to refer to any form or analogue of IFN-β that retains the biological activity of native IFN-β and preferably retains the activity of IFN-β as present in the lung and in particular the pulmonary epithelium when induced by viral infection such as influenza or rhinovirus infection.

The IFN-β may be identical to or comprise the sequence of human IFN-β1a or human IFNβ-1b. However, the IFN-β may also be a variant to such a native sequence, for example, a variant having at least 80%, at least 85%, at least 90%, at least 95-99% identity. It may have one or more chemical modifications provided the desired biological activity is retained.

The IFN-β will preferably be a recombinant IFN-β, e.g. produced in cells in vitro by expression of the polypeptide from a recombinant expression vector and purified from such culture.

Preferred is human recombinant IFN-β1a, e.g. as available from Rentschler Biopharma SE or Akron Biotechnology, LLC (Akron Biotech).

Formulation and Mode of Administration

The IFN-β for administration by inhalation will generally be formulated as an aqueous solution, preferably at or about neutral pH, e.g. about pH 6-7, preferably, for example pH 6.5. Methods for formulating IFN-β for airway delivery in aqueous solution are well known, see for example U.S. Pat. No. 6,030,609 and European Patent no. 2544705. Preferably such an aqueous formulation will be employed which does not contain mannitol, human serum albumin (HSA) and arginine which are present in injectable IFN-β formulations. The composition may preferably contain an antioxidant such as methionine, e.g. DL-methionine. Such a ready-to-use formulation of IFN-β1a can also be obtained commercially, e.g. prepared in syringes at appropriate dilution of the IFN-β, e.g. from Vetter Pharma. It may conform with the formulation designated herein as SNG001 as previously used in clinical trials as referred to above in patients exhibiting viral exacerbation of asthma or COPD (subject to possible variation of the precise IFN-β1a concentration). Further details of this formulation are available in European Patent no. 2544705 and in the exemplification herein below. The concentration of IFN-β1a may be adjusted as discussed below. The precise preferred concentration of IFN-β, or more particularly IFN-β1a, may vary with the precise mode of delivery.

A pH neutral or about neutral pH IFN-β formulation e.g. pH 6.5, rather than a lower pH formulation, is especially favoured. A low pH is known to trigger cough.

Delivery may be made using any device for aerosolization of a liquid formulation which retains the IFN-β activity, e.g. a nebuliser. Various nebulizers for drug delivery are commercially available and might be employed, e.g. the I-neb or Ultra nebuliser made by Philips Respironics and Aerogen respectively. Both devices have been shown to enable convenient inhalation delivery of IFN-β1a with retention IFN-β activity after aerosolization.

Dosage

A suitable IFN-β dose for any inhalation delivery mode may be established by a dose escalation study with assessment of induced anti-viral response in the lungs e.g. sputum cell gene expression of MX1, generally a dose which ensures a robust anti-viral response within 24 hours after dose administration, preferably so as to support a once-a-day dosing regimen. This may be assessed by reference to appropriate biomarkers.

For nebuliser delivery of an aqueous formulation containing IFN-β1a, an aqueous formulation as discussed above contains about, but not limited to, 3 to 16 MIU/ml IFN-β1a. Preferably the concentration is 11-13 MIU/ml IFN-β1a. More preferably, 11-12 MIU/ml may be found suitable.

A suitable once-a-day dosing schedule has been achieved by delivering 0.5 ml or, preferably about 0.25 to 1.3 ml, of an aqueous formulation containing IFN-β1a at about 3 to 16 MIU/ml, preferably 11-12 MIU/ml, more preferably 12 MIU/ml IFN-β1a, from the I-neb nebuliser (Phillips Respironics) and may be found suitable with other nebulisers providing similar efficiency of airway delivery. If using alternative nebulisers, the concentration of IFN-β1a in the formulation and the dose or volume may need to be adjusted to take into account differences in efficiency of drug delivery to the lungs. For example, the Ultra nebuliser (Aerogen) delivers a lower percentage of the emitted dose to the lungs. To account for this a dose of 0.65 ml to 1.3 ml of a 12 MIU/ml IFN-β1a aqueous solution is preferred. Once daily delivery may preferably be carried out. Delivery may be over a number of days, e.g. for 3 or more days, for 5 or more days or 7 or more days, e.g. up to 14 days to alleviate LRT illness and preferably step improvement in score back to a lower score.

Timing of Administration

Administration of IFN-β is recommended in COPD patients undergoing treatment with systemic corticosteroids for worsening of COPD symptoms and which patients are infected with virus preferably within 2 days of worsening of symptoms.

Thus in accordance with the invention, IFN-β, e.g. recombinant IFN-β1a, may be administered by inhalation to a patient with a virus infection preferably within 2 days of worsening symptoms. Use may encompass patients more than 2 days post onset of worsening of symptoms e.g. 3, 4, 5, 6, 7, 8, or 9 days or more post onset of symptoms of viral infection.

Effectiveness of treatment may be assessed daily by assessing Peak Expiratory Flow Rate.

Combination Therapy

Although the data now presented support use of inhaled IFN-β as an add on therapeutic agent to reduce the severity of virus-induced exacerbations in COPD patients undergoing systemic corticosteroid treatment, it will be appreciated that such administration of IFN-β is not excluded with one or more other therapeutic agents which may assist improvement of one or more symptoms of the patient arising from the viral infection. Use of inhaled IFN-β may be combined for example with administration of an antibiotic or antiviral therapy proposed for preventing or reducing the severity of LRT illness in COPD patients whose symptoms are exacerbated by viral infection. Such combined therapy may involve simultaneous, sequential or separate administration of IFN-β and another therapeutic agent as appropriate.

Method of Treatment

In another aspect, the invention provides a method of reducing the severity of lower respiratory tract illness in a COPD patient treated with systemic corticosteroid but also infected with a virus capable of causing respiratory infection and/or improving one or more symptoms and/or outcome in a patient so infected, wherein said method comprises administering IFN-β by inhalation. The IFN-β may be administered as an add on therapeutic agent, alone or in combination with one or more further therapeutic agents to assist improvement of one or more symptoms arising from the same viral infection as discussed above.

### EXAMPLE

Summary of Protocol to Investigate the Efficacy of IFN-β in the Treatment of Virus-Induced Exacerbations of COPD Patients Treated with Systemic Corticosteroids.

The Applicant has developed an inhaled formulation of IFN-β1a (SNG001) for use in viral exacerbations of COPD in patients being treated systemically with corticosteroids. The purpose of this study was to confirm IFN-β driven antiviral biomarker up-regulation and to assess clinical effects in COPD patients either with or without respiratory virus-induced exacerbations following the administration of inhaled SNG001, and to investigate how the use of systemic corticosteroid administration affects the antiviral activity of inhaled IFN-β in the lung.

SNG001 is a solution of IFN-β1a at a concentration of 12 MIU/mL. The drug substance, recombinant IFN-β1a, and the finished product are manufactured by either Rentschler Biotechnologie GmbH, Erwin-Rentschler-Straβe 21, 88471 Laupheim, Germany or by Vetter Development Services USA Inc, 8025 Lamon Ave, Skokie, Ill. 60077, USA.

The study medication is presented as a ready-to-use aqueous solution at pH 6.5.

Study Design

The study was divided into two parts. During Part 1, the local tolerance of inhaled IFN-β (SNG001) and lung antiviral biomarker responses were assessed in COPD patients without symptoms of a respiratory viral infection. In part 2, the clinical effect of inhaled IFN-β and lung antiviral biomarkers responses were assessed in COPD patients with a confirmed respiratory viral infection.

Part 1: Ten COPD patients (in a stable condition without symptoms of a respiratory viral infection) received SNG001 (6 MIU IFN-β1a) or placebo, via a CE marked breath-actuated nebuliser (I-neb Philips Respironics), once daily for 3 days. Patients were randomised in a 4:1 ratio respectively. Hence, 8 patients were randomised to SNG001 and 2 to placebo. Assessment including lung function testing, vital signs, blood and sputum sampling, adverse event (AE) and concomitant medication reporting were performed during the study. Sputum samples were collected 24 hours after the first and third dose for biomarker assessment. Sputum cell gene expression of IFN-β dependent antiviral biomarkers, including MX1, was determined by reverse transcription quantitative PCR.

Part 2: Treatment of COPD patients with a confirmed respiratory virus infection.

COPD patients, who developed upper respiratory virus symptoms (cold symptoms) and/or had a deterioration in their COPD symptoms, were tested for the presence of a common respiratory virus. This test involved obtaining a nose and throat swab and/or a sputum sample (optional) from the patient. The nose and throat swab was sufficient for the test. The presence of respiratory virus was established using multiplex PCR technology, such the BioFire FilmArray system available from BioMerieux.

If the respiratory panel virus test was positive the patient was randomised to study treatment and stratified to one of two groups according to whether they had cold symptoms and/or a deterioration in COPD symptoms without moderate COPD exacerbation (Group A) or had a moderate COPD exacerbation with or without cold symptoms (Group B). For the purposes of this study a moderate exacerbation is defined according to the GOLD 2017 guidelines as ‘an acute worsening of respiratory symptoms that results in additional therapy treated with short acting bronchodilators (SABDs) plus antibiotics and/or oral corticosteroids’.

Patients were eligible for Group B only if the exacerbation of their COPD symptoms required treatment with oral corticosteroids and/or antibiotics. If the patient did not require treatment with oral corticosteroids and/or antibiotics, they did not meet the criteria for moderate exacerbation and were stratified to Group A only.

Patients were randomised 1:1 to receive SNG001 (6 MIU IFN-β1a) or placebo once daily for 14 days. Doses were delivered in the clinic and at home, via a CE-marked breath-actuated nebuliser (I-neb Philips Respironics). The first dose of study medication was administered within 48 hours of the onset of respiratory virus symptoms and/or deterioration in COPD symptoms (Group A) or the onset of a moderate COPD exacerbation requiring treatment with a systemic corticosteroid and/or antibiotics (Group B).

To assess the effectiveness of treatment Peak Expiratory Flow Rate (PEFR), a measure of lung function, was assessed daily throughout the treatment period.

Sputum samples were collected where possible at clinic visits to assess lung antiviral responses to treatment. Sputum cell gene expression of IFN-β dependent antiviral biomarkers (including Mx1) was determined by reverse transcription quantitative PCR.

Formulation of recombinant IFN-β1a

The formulation (referred to as SNG001) provides recombinant IFN-β1a (manufactured by Rentschler Biopharma SE or Akron Biotechnology, LLC) formulated as an aqueous solution buffered at pH 6.5. The composition is set out in the table below. Unlike some other commercial preparations, it does not contain mannitol, human serum albumin or arginine. The formulation is provided in ready-to-use syringes by Vetter Pharma, Catalent Inc. or Patheon N.V.

SNG001 formulation:

| Ingredient | Quantity (per ml) | | Function |
|---|---|---|---|
| IFN-β1a | About 12 | MIU/ml | Active ingredient |
| Sodium dihydrogen | 5.92 | mg | Buffer component |
| phosphate dihydrate | | | |
| Disodium phosphate | 2.13 | mg | Buffer component |
| dihydrate | | | |
| Sodium citrate | 20.58 | mg | Chelating agent, buffer |
| | | | component |
| Methionine | 0.30 | mg | Stabiliser, antioxidant |
| Water | 1 | ml | Solvent |

Peak Expiratory Flow Rate Test

PEFR is measured with a peak flow meter, a hand-held device used to monitor a person's ability to breathe out air. An example of a suitable PFER monitor is the eMini-Wright, Digital Peak Flow Meter (model. 3210001) made by Clement Clarke International.

Findings Supporting Benefit of IFN-β Administration to COPD Patients with Viral Exacerbations and Who are Taking Corticosteroid Medication

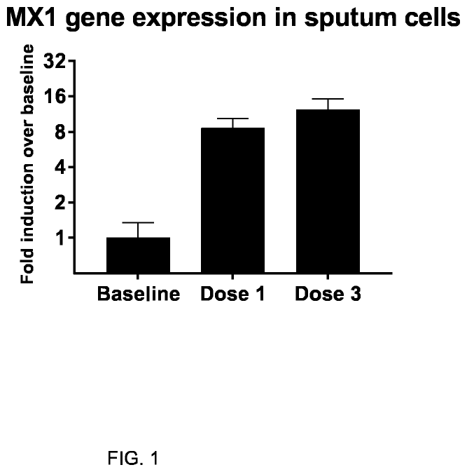

In Part 1 of the study, conducted in COPD patients without symptoms of a respiratory viral infection and not treated with systemic corticosteroids, IFN-β was shown to be well tolerated via the inhaled route. Gene expression of the IFN-β-dependent antiviral biomarker MX1 was elevated and maintained in sputum cells 24 hours post first and third doses demonstrating that inhalable IFN-β can switch on and maintain antiviral defences in the lungs of COPD patients (FIG. 1).

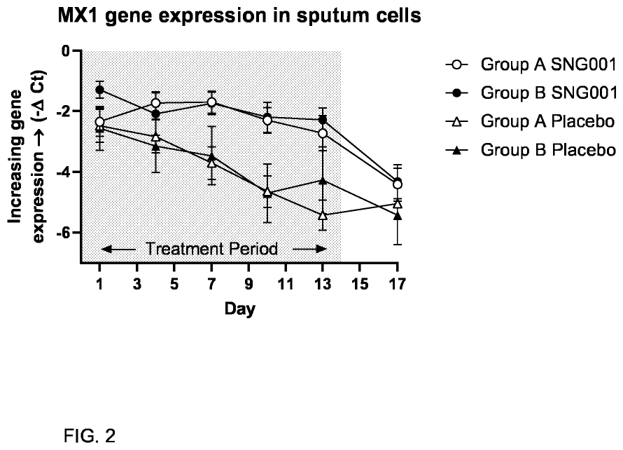

In part 2 of the study, conducted in COPD patients with a confirmed respiratory viral infection, inhaled IFN-β was shown to be well tolerated via the inhaled route. Over the treatment period, IFN-β (compared to placebo) significantly enhanced patients' lung antiviral responses to viral infection, assessed by measuring statistically significant increases in IFN-β-dependent antiviral biomarkers such as MX1 (p=<0.001) in lung (sputum) cells. Biomarker responses were similar in patients in Group A and Group B, showing that treatment with systemic corticosteroids did not suppress lung antiviral responses to inhalable IFN-β. This was further evidenced by the fact that patients in Group B, requiring treatment with systemic corticosteroids and/or antibiotics at the start of the treatment period for an exacerbation, had significantly better lung function during the treatment period (difference in change from baseline in morning PEFR between patients receiving IFN-β and placebo over Days 2-15 was 25.5 L/min [95% Cl 1.1, 49.9]; p=0.041; FIG. 2). In summary, inhalable IFN-β boosted lung antiviral responses and showed clinical benefit in COPD patients treated with systemic corticosteroids for a virus-induced exacerbation.

1. A method for treating virus-induced Chronic Obstructive Pulmonary Disease (COPD) exacerbations in patients treated with a systemic corticosteroid, wherein the interferon-beta (IFN-β) is administered by inhalation.

2. A method according to claim 1, wherein said patient is infected with rhinovirus, influenza, respiratory syncytial virus (RSV), adenovirus, parainfluenza, human metapneumovirus or coronavirus and wherein the coronavirus is not an highly pathogenic coronavirus that causes severe acute respiratory syndrome (SARS), Middle East respiratory syndrome (MERS) or coronavirus disease of 2019 (COVID-19).

3. A method according to claim 2, wherein said virus is rhinovirus or influenza.

4. A method according to claim 1, wherein the IFN-β is recombinant human IFN-β1a.

5. A method according to claim 1, wherein the IFN-β is formulated in an aqueous solution at about pH 6-7.

6. A method according to claim 1, wherein administration by inhalation is to the patient's airways and comprises aerosolization of a liquid formulation of the IFN-β.

7. A method according to claim 6, wherein administration is by use of a nebuliser.

8. A method according to claim 1, wherein administration of the IFN-β is as a single inhaled dose daily.

9. A method according to claim 1, wherein the patient is undergoing systemic treatment with a corticosteroid selected from the group consisting of: prednisolone, hydrocortisone, dexamethasone, methylprednisolone and prednisone or combinations thereof.

10. A method according to claim 1, wherein the IFN-β is administered by inhalation in combination with administration of one or more further therapeutic agents to assist improvement of one or more symptoms arising from the same viral infection, wherein each further agent is administered simultaneously, separately or sequentially.

11. The method of claim 5, wherein the IFN-β is formulated in an aqueous solution at pH 6.5.

12. The method of claim 5, wherein the IFN-β is formulated to omit mannitol, human serum albumin and arginine.
